(19) United States
(12) Patent Application Publication
(10) Pub. No.: US 2025/0292401 A1
(43) Pub. Date: Sep. 18, 2025

(54) IMAGE PROCESSING DEVICE, ENDOSCOPE SYSTEM, IMAGE PROCESSING METHOD, AND PROGRAM

(71) Applicant: FUJIFILM CORPORATION, Tokyo (JP)

(72) Inventor: Toshihiro USUDA, Kanagawa (JP)

(21) Appl. No.: 19/047,630

(22) Filed: Feb. 7, 2025

(30) Foreign Application Priority Data
Mar. 12, 2024 (JP) 2024-038215

(51) Int. Cl. G06T 7/00; A61B 8/00; A61B 8/06; A61B 8/12; G06V 10/70

(52) U.S. Cl. CPC G06T 7/0012; A61B 8/06; A61B 8/12; A61B 8/488; A61B 8/5246; G06V 10/70; G06T 2207/10132; G06T 2207/20081; G06T 2207/30096; G06T 2207/30101; G06V 2201/031

(57) ABSTRACT

The image processing device includes a processor. The processor acquires an ultrasound image which is obtained by a B-mode method in an ultrasound diagnosis for a subject and in which an observation target region of the subject is shown, and blood flow distribution information obtained by a Doppler method used in combination with the B-mode method in the ultrasound diagnosis. The processor outputs a recognition result obtained by executing recognition processing of inputting the ultrasound image and the blood flow distribution information to a trained model to cause the trained model to recognize the observation target region.

### CROSS-REFERENCE TO RELATED APPLICATIONS

This application claims priority under 35 USC 119 from Japanese Patent Application No. 2024-038215, filed on Mar. 12, 2024, the disclosure of which is incorporated by reference herein.

### BACKGROUND

### 1. Technical Field

The present disclosure relates to an image processing device, an endoscope system, an image processing method, and a program.

### 2. Related Art

JP2016-047474A discloses an ultrasound diagnostic device comprising a transmission control unit, a detection unit, an evaluation unit, and a display image control unit. In the ultrasound diagnostic device described in JP2016-047474A, the transmission control unit transmits first ultrasound to a biological tissue from an ultrasound probe, and transmits second ultrasound to the ultrasound probe after the first ultrasound is transmitted. In the ultrasound diagnostic device described in JP2016-047474A, the detection unit detects the presence or absence of movement of an object in the biological tissue by the first ultrasound in a two-dimensional region based on an echo signal obtained by the transmission of the second ultrasound. In the ultrasound diagnostic device described in JP2016-047474A, the evaluation unit evaluates tissue properties for a portion of interest of the biological tissue based on the detection by the detection unit. In the ultrasound diagnostic device described in JP2016-047474A, the display image control unit displays an image according to the evaluation by the evaluation unit. The processing of detecting the presence or absence of movement of the object by the detection unit is color Doppler processing or B-flow processing, and the evaluation unit performs evaluation according to a size of a region in which Doppler data is obtained by the color Doppler processing or a region in which B-flow data is obtained by the B-flow processing.

WO2022/196088A discloses an ultrasound diagnostic device comprising an image acquisition unit, a monitor, a debris specifying unit, and a debris movement information providing unit. In the ultrasound diagnostic device described in WO2022/196088A, the image acquisition unit acquires an ultrasound image obtained by imaging a cyst of a subject by transmitting and receiving ultrasound to and from the subject. In the ultrasound diagnostic device described in WO2022/196088A, the monitor displays the ultrasound image. In the ultrasound diagnostic device described in WO2022/196088A, the debris specifying unit specifies a debris in the cyst from each of a first ultrasound image acquired by the image acquisition unit and a second ultrasound image acquired by the image acquisition unit by imaging the cyst in the same direction as a direction when the first ultrasound image is captured after an external force different from an external force at the time of capturing the first ultrasound image acts on the cyst or in a state where the external force is acting on the cyst. In the ultrasound diagnostic device described in WO2022/196088A, the debris movement information providing unit provides information regarding movement of the debris in the cyst specified by the debris specifying unit between the first ultrasound image and the second ultrasound image.

In the ultrasound diagnostic device described in WO2022/196088A, the debris specifying unit specifies the debris in a state in which the cyst specified from the first ultrasound image and the cyst specified from the second ultrasound image are aligned with each other. In the ultrasound diagnostic device described in WO2022/196088A, the debris specifying unit includes a sameness determination unit that determines that the same cyst is imaged by performing image recognition on each of the first ultrasound image and the second ultrasound image. In the ultrasound diagnostic device described in WO2022/196088A, the sameness determination unit determines that the same cyst is imaged by a trained determination model with the first ultrasound image and the second ultrasound image as inputs.

JP2012-061075A discloses an ultrasound diagnostic device comprising a physical quantity calculation unit, an elastic image creation unit, and a detection unit. In the ultrasound diagnostic device described in JP2012-061075A, the physical quantity calculation unit calculates a physical quantity related to elasticity in each part of a subject. In the ultrasound diagnostic device described in JP2012-061075A, the elastic image creation unit creates an elastic image of the subject based on the physical quantity calculated by the physical quantity calculation unit. In the ultrasound diagnostic device described in JP2012-061075A, the elastic image creation unit creates the elastic image based on the physical quantity using a statistical feature of the physical quantity calculated excluding a liquid portion in the subject. In the ultrasound diagnostic device described in JP2012-061075A, the detection unit detects the liquid portion based on an echo signal for ultrasound transmitted to the subject. In the ultrasound diagnostic device described in JP2012-061075A, the detection unit is a fluid information acquisition unit that acquires fluid information based on the echo signal. In the ultrasound diagnostic device described in JP2012-061075A, the fluid information is any one of color Doppler data, B-flow data, and harmonic data included in the echo signal, which are acquired based on the echo signal.

### SUMMARY

One embodiment according to the present disclosure provides an image processing device, an endoscope system, an image processing method, and a program capable of outputting a highly accurate recognition result for an observation target region shown in an ultrasound image obtained by a B-mode method.

A first aspect according to the present disclosure relates to an image processing device comprising: a processor, in which the processor acquires an ultrasound image which is obtained by a B-mode method in an ultrasound diagnosis for a subject and in which an observation target region of the subject is shown, and blood flow distribution information obtained by a Doppler method used in combination with the B-mode method in the ultrasound diagnosis, and outputs a recognition result obtained by executing recognition processing of inputting the ultrasound image and the blood flow distribution information to a trained model to cause the trained model to recognize the observation target region.

A second aspect according to the present disclosure relates to the image processing device according to the first aspect, in which the observation target region is a lesion, and the recognition result is information capable of distinguishing the lesion from a portion other than the lesion.

A third aspect according to the present disclosure relates to the image processing device according to the second aspect, in which the lesion is a cyst.

A fourth aspect according to the present disclosure relates to the image processing device according to the second or third aspect, in which the trained model is obtained by performing machine learning for recognizing the lesion or machine learning for recognizing the lesion and a blood vessel shown in the ultrasound image.

A fifth aspect according to the present disclosure relates to the image processing device according to the first or second aspect, in which the observation target region is a blood vessel, and the recognition result is information capable of distinguishing the blood vessel from a portion other than the blood vessel.

A sixth aspect according to the present disclosure relates to the image processing device according to the fifth aspect, in which the trained model is obtained by performing machine learning for recognizing the blood vessel or machine learning for recognizing a lesion shown in the ultrasound image and the blood vessel shown in the ultrasound image.

A seventh aspect according to the present disclosure relates to the image processing device according to any one of the first to sixth aspects, in which the blood flow distribution information is a map capable of specifying an intensity of a blood flow.

An eighth aspect according to the present disclosure relates to the image processing device according to any one of the first to seventh aspects, in which the outputting of the recognition result includes displaying the recognition result on a first screen.

A ninth aspect according to the present disclosure relates to the image processing device according to any one of the first to eighth aspects, in which the ultrasound image is displayed on a second screen in a foreground, and the recognition processing is executed in a background.

A tenth aspect according to the present disclosure relates to the image processing device according to any one of the first to ninth aspects, in which, in a case where the recognition processing is being executed, the processor outputs information indicating that the recognition processing is being executed.

An eleventh aspect according to the present disclosure relates to an endoscope system comprising: the image processing device according to any one of the first to tenth aspects; and an ultrasound probe that emits ultrasound and detects a reflected wave of the ultrasound in a state in which the ultrasound probe is inserted into a body of the subject, in which the ultrasound image and the blood flow distribution information are generated based on the reflected wave detected by the ultrasound probe.

A twelfth aspect according to the present disclosure relates to an image processing method comprising: acquiring an ultrasound image which is obtained by a B-mode method in an ultrasound diagnosis for a subject and in which an observation target region of the subject is shown, and blood flow distribution information obtained by a Doppler method used in combination with the B-mode method in the ultrasound diagnosis; and outputting a recognition result obtained by executing recognition processing of inputting the ultrasound image and the blood flow distribution information to a trained model to cause the trained model to recognize the observation target region.

A thirteenth aspect according to the present disclosure relates to a program for causing a computer to execute a process comprising: acquiring an ultrasound image which is obtained by a B-mode method in an ultrasound diagnosis for a subject and in which an observation target region of the subject is shown, and blood flow distribution information obtained by a Doppler method used in combination with the B-mode method in the ultrasound diagnosis; and outputting a recognition result obtained by executing recognition processing of inputting the ultrasound image and the blood flow distribution information to a trained model to cause the trained model to recognize the observation target region.

### BRIEF DESCRIPTION OF THE DRAWINGS

Exemplary embodiments of the technology of the disclosure will be described in detail based on the following figures, wherein:

FIG. 1 is a conceptual diagram showing an example of an aspect in which an endoscope system is used;

FIG. 2 is a conceptual diagram showing an example of an overall configuration of the endoscope system;

FIG. 3 is a block diagram showing an example of a configuration of an ultrasound endoscope;

FIG. 4 is a conceptual diagram showing an example of a processing content of a generation unit;

FIG. 5 is a conceptual diagram showing an example of an aspect in which a cyst recognition model is generated by causing a model to learn a learning image group;

FIG. 6 is a conceptual diagram showing an example of processing contents of a recognition unit and a control unit;

FIG. 7 is a flowchart showing an example of a flow of diagnosis support processing;

FIG. 8 is a flowchart showing an example of a flow of background processing;

FIG. 9 is a flowchart showing an example of a flow of foreground processing;

FIG. 10 is a conceptual diagram showing an example of an aspect in which a blood vessel recognition model is generated by causing a model to learn a learning image group;

FIG. 11 is a conceptual diagram showing a modification example of processing contents of the recognition unit and the control unit;

FIG. 12 is a flowchart showing a modification example of the flow of the diagnosis support processing;

FIG. 13 is a conceptual diagram showing an example of an aspect in which information indicating that cyst recognition processing is being executed is displayed on a screen; and

FIG. 14 is a conceptual diagram showing an example of a series of pieces of processing in which a processor included in a computer gives a processing execution request to an external device via a network, the external device executes processing according to the processing execution request, and the processor included in the computer receives a processing result from the external device.

### DETAILED DESCRIPTION

Hereinafter, an example of embodiments of an image processing device, an endoscope system, an image processing method, and a program according to the present disclosure will be described with reference to the accompanying drawings.

First, terms used in the following description will be described.

CPU is an abbreviation for “central processing unit”. GPU is an abbreviation for “graphics processing unit”. TPU is an abbreviation for “tensor processing unit”. RAM is an abbreviation for “random-access memory”. EEPREEPROM is an abbreviation for “electrically erasable programmable read-only memory”. ASIC is an abbreviation for “application-specific integrated circuit”. PLD is an abbreviation for “programmable logic device”. FPGA is an abbreviation for “field-programmable gate array”. SoC is an abbreviation for “system-on-a-chip”. SSD is an abbreviation for “solid-state drive”. USB is an abbreviation for “Universal Serial Bus”. HDD is an abbreviation for “hard disk drive”. EL is an abbreviation for “electro-luminescence”. CMOS is an abbreviation for “complementary metal-oxide-semiconductor”. CCD is an abbreviation for “charge-coupled device”. LAN is an abbreviation for “local area network”. WAN is an abbreviation for “wide area network”. AI is an abbreviation for “artificial intelligence”. BLI is an abbreviation for “blue light imaging”. LCI is an abbreviation for “linked color imaging”. NN is an abbreviation for “neural network”. CNN is an abbreviation for “convolutional neural network”. R-CNN is an abbreviation for “region based convolutional neural network”. YOLO is an abbreviation for “you only look once”. RNN is an abbreviation for “recurrent neural network”. FCN is an abbreviation for “fully convolutional network”. FIFO is an abbreviation for “first in first out”.

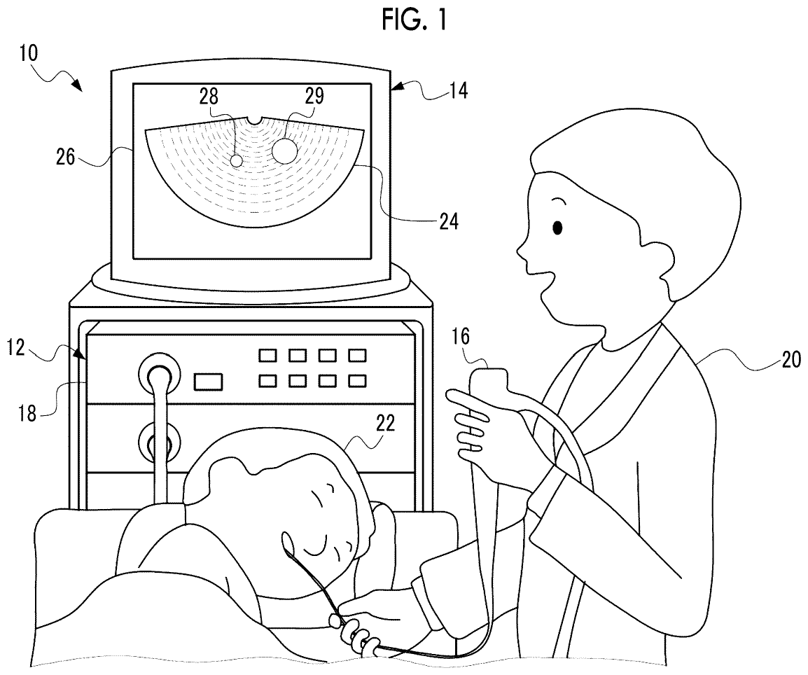

As shown in FIG. 1 as an example, an endoscope system 10 comprises an ultrasound endoscope 12 and a display device 14. The ultrasound endoscope 12 is a convex type ultrasound endoscope, and comprises an ultrasound endoscope body 16 and a processing device 18. In the present embodiment, the endoscope system 10 is an example of an “endoscope system” according to the present disclosure. In addition, in the present embodiment, the processing device 18 is an example of an “image processing device” according to the present disclosure.

It should be noted that, in the present embodiment, although the convex type ultrasound endoscope is shown as an example of the ultrasound endoscope 12, this is merely an example, and the present disclosure is established even in a case of a radial type ultrasound endoscope. In addition, in the present embodiment, although the ultrasound endoscope 12 is shown as an example, this is merely an example, and the present disclosure is established even in a case of an extracorporeal ultrasound diagnostic device.

The endoscope system 10 is used by a user 20. An example of the user 20 is a doctor. The ultrasound endoscope body 16 is connected to the processing device 18, and transmits and receives various signals to and from the processing device 18. That is, the processing device 18 outputs a signal to the ultrasound endoscope body 16 to control an operation of the ultrasound endoscope body 16, or performs various types of signal processing on a signal input from the ultrasound endoscope body 16.

The ultrasound endoscope 12 is an endoscope used for ultrasound diagnosis on a subject 22 (for example, a patient). In the ultrasound diagnosis on the subject 22, in a case where the user 20 observes an internal part (for example, an internal organ such as a pancreas or gallbladder) that is a part present inside a body of the subject 22, the user 20 inserts the ultrasound endoscope body 16 into the body of the subject 22 from a mouth or a nose of the subject 22 (in the example shown in FIG. 1, the mouth). Then, in a state in which the ultrasound endoscope body 16 is inserted into the body of the subject 22, the ultrasound endoscope body 16 emits ultrasound to the internal part at a predetermined position (for example, a position of a stomach or a duodenum) of the body of the subject 22, and detects a reflected wave obtained by the emitted ultrasound being reflected at the internal part. The processing device 18 generates an ultrasound image 24 based on the reflected wave detected by the ultrasound endoscope body 16, and outputs the ultrasound image 24 to the display device 14 or the like.

It should be noted that the example shown in FIG. 1 shows an aspect in which an upper gastrointestinal endoscopy is performed, but the present disclosure is not limited to this, and the present disclosure can also be applied to a lower gastrointestinal endoscopy, a bronchoscopy, and the like.

The display device 14 displays various types of information including an image under the control of the processing device 18. Examples of the display device 14 include a liquid crystal display and an EL display. The ultrasound image 24 generated by the processing device 18 is displayed as a moving image on a screen 26 of the display device 14. In the example shown in FIG. 1, a cyst 28 and a blood vessel 29 are shown in the ultrasound image 24 displayed on the screen 26. In the present embodiment, the screen 26 is an example of a “first screen” and a “second screen” according to the present disclosure.

It should be noted that the example shown in FIG. 1 shows a form example in which the ultrasound image 24 is displayed on the screen 26 of the display device 14, but this is merely an example, and the ultrasound image 24 may be displayed on a display device (for example, a display of a tablet terminal) other than the display device 14. The ultrasound image 24 may be stored in a computer-readable non-transitory storage medium (for example, a flash memory, an HDD, and/or a magnetic tape).

The display device 14 displays an image according to a type of an ultrasound diagnostic method, which is a diagnostic method using ultrasound. A type of an ultrasonography method is selected by the user 20 or is selected according to various conditions. Examples of the type of the ultrasonography method include a brightness mode (B-mode) method and a Doppler method (for example, a color Doppler method).

The B-mode method is an ultrasound diagnosis method that generates and displays a B-mode image. The B-mode image refers to a two-dimensional tomographic image obtained in a cross section parallel to a direction of ultrasound propagation and is obtained by converting an intensity of the reflected wave, obtained by the ultrasound emitted to the internal part being reflected at the internal part, into a brightness on the screen 26.

The Doppler method is an ultrasound diagnosis method that generates and displays a Doppler image. The Doppler image refers to an image that shows blood dynamics in a blood vessel with color information by using the reflected wave, obtained by the ultrasound emitted to the internal part being reflected at the internal part. The image showing the blood dynamics in the blood vessel is, for example, an image expressed by red-based and blue-based colors. The red-based color is a color indicating a blood flow toward a side where the ultrasound is emitted, and the blue-based color is a color indicating a blood flow away from the side where the ultrasound is emitted. In a case where a reflected wave having a higher frequency than the ultrasound that hits the blood flow is obtained, the blood flow is specified as a blood flow directed toward the side where the ultrasound is emitted, whereas in a case where a reflected wave having a lower frequency than the ultrasound that hits the blood flow is obtained, the blood flow is specified as a blood flow directed away from the side where the ultrasound is emitted. In the blood vessel, there are a portion where the blood flow is fast and a portion where the blood flow is slow, and in the Doppler image, the speed of the blood flow is expressed as a gradation through color mapping.

On the screen 26, the B-mode image or the Doppler image is displayed as the ultrasound image 24, or the B-mode image and the Doppler image are displayed in parallel, according to the ultrasound diagnostic method selected by the user 20. In the example shown in FIG. 1, the B-mode image is displayed on the screen 26 as the ultrasound image 24. The ultrasound image 24 is a moving image including a plurality of frames generated according to a predetermined frame rate (for example, 30 frames/second). A frame rate of the Doppler method is lower than a frame rate of the B-mode method. An example of the frame rate of the Doppler method (that is, the frame rate at which the Doppler image is generated) is 15 frames/second. Here, although the moving image is shown as an example, this is merely an example, and the present disclosure is established even in a case where the ultrasound image 24 is a still image. In the following description, in order to facilitate understanding of the present disclosure, the description will be made on the premise that the ultrasound image 24 is the B-mode image.

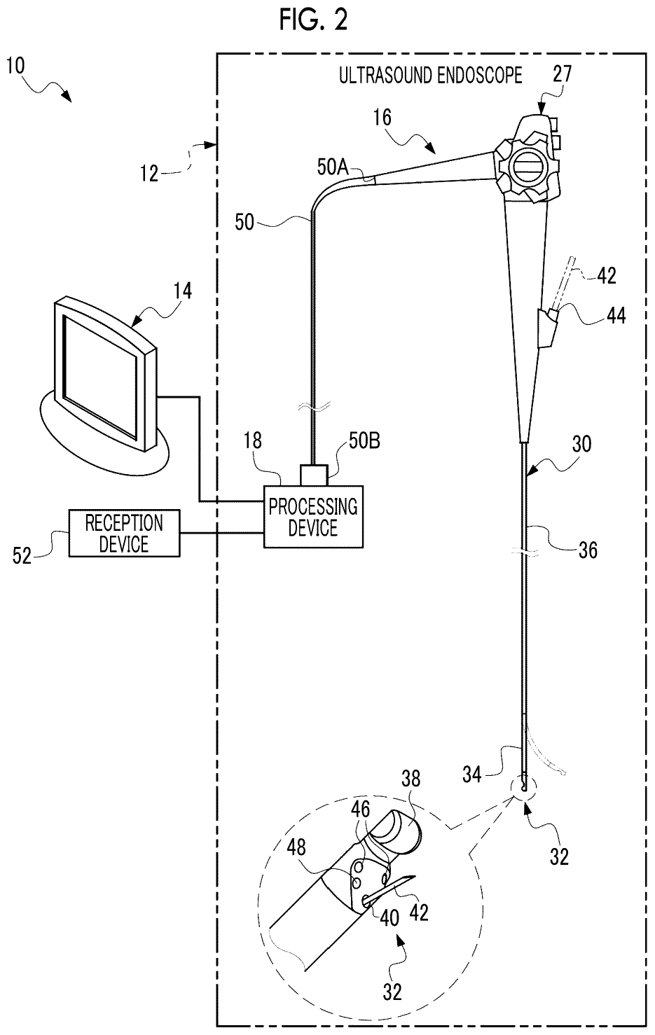

As shown in FIG. 2 as an example, the ultrasound endoscope body 16 comprises an operating part 27 and an insertion part 30. The insertion part 30 is formed in a tubular shape. The insertion part 30 includes a distal end part 32, a bendable part 34, and a flexible part 36. The distal end part 32, the bendable part 34, and the flexible part 36 are disposed in an order of the distal end part 32, the bendable part 34, and the flexible part 36 from a distal end side to a base end side of the insertion part 30. The flexible part 36 is formed of an elongated flexible material and connects the operating part 27 and the bendable part 34. The bendable part 34 is partially bent or rotated about an axial center of the insertion part 30 by operating the operating part 27. As a result, the insertion part 30 is sent to a back side of a luminal organ while being bent in accordance with a shape of the luminal organ (for example, a shape of a duodenal tract) or being rotated about an axis of the insertion part 30.

The distal end part 32 is provided with an ultrasound probe 38 and a treatment tool opening 40. The ultrasound probe 38 is provided on a distal end side of the distal end part 32. The ultrasound probe 38 is a convex type ultrasound probe, and in a state of being inserted into the body of the subject 22, emits the ultrasound to the internal part and receives the reflected wave, obtained by the emitted ultrasound being reflected at the internal part.

The treatment tool opening 40 is formed on a base end side of the distal end part 32 with respect to the ultrasound probe 38. The treatment tool opening 40 is an opening for allowing a treatment tool 42 to protrude from the distal end part 32. A treatment tool insertion port 44 is formed at the operating part 27, and the treatment tool 42 is inserted into the insertion part 30 through the treatment tool insertion port 44. The treatment tool 42 passes through the insertion part 30 and protrudes from the treatment tool opening 40 to an outside of the ultrasound endoscope body 16. The treatment tool opening 40 also functions as a suction port for suctioning blood, internal waste, and the like.

In the example shown in FIG. 2, a puncture needle is shown as the treatment tool 42. It should be noted that this is merely an example, and the treatment tool 42 may be forceps and/or a cannula.

An illumination device 46 and a camera 48 are provided in the distal end part 32. The illumination device 46 applies light. Examples of a type of light applied from the illumination device 46 include visible light (for example, white light), invisible light (for example, near-infrared light), and/or special light. Examples of the special light include light for BLI and/or light for LCI.

The camera 48 images an inside of the luminal organ by an optical method. A CMOS image sensor is used in the camera 48. The CMOS image sensor is merely an example, and other types of image sensors such as a CCD image sensor may be used. The image obtained by being captured by the camera 48 is displayed on the display device 14, is displayed on a display device (for example, a display of a tablet terminal) other than the display device 14, is stored in a storage medium (for example, a flash memory, an HDD, and/or a magnetic tape), or is stored in an electronic medical record.

The ultrasound endoscope 12 comprises the processing device 18 and a universal cord 50. The universal cord 50 has a base end part 50A and a distal end part 50B. The base end part 50A is connected to the operating part 27. The distal end part 50B is connected to the processing device 18. That is, the ultrasound endoscope body 16 and the processing device 18 are connected to each other via the universal cord 50.

The endoscope system 10 comprises a reception device 52. The reception device 52 is connected to the processing device 18. The reception device 52 receives an instruction from a user. Examples of the reception device 52 include an operation panel having a plurality of hard keys and/or a touch panel, a keyboard, a mouse, a trackball, a foot switch, a smart device, and/or a microphone.

The processing device 18 performs various types of signal processing or transmits and receives various signals to and from the ultrasound endoscope body 16 and the like in response to the instruction received by the reception device 52. For example, the processing device 18 emits the ultrasound to the ultrasound probe 38 in response to the instruction received by the reception device 52, generates the ultrasound image 24 (see FIG. 1) based on the reflected wave received by the ultrasound probe 38, and outputs the generated ultrasound image 24.

The display device 14 is also connected to the processing device 18. The processing device 18 controls the display device 14 in response to the instruction received by the reception device 52. As a result, for example, the ultrasound image 24 generated by the processing device 18 is displayed on the screen 26 of the display device 14 (see FIG. 1).

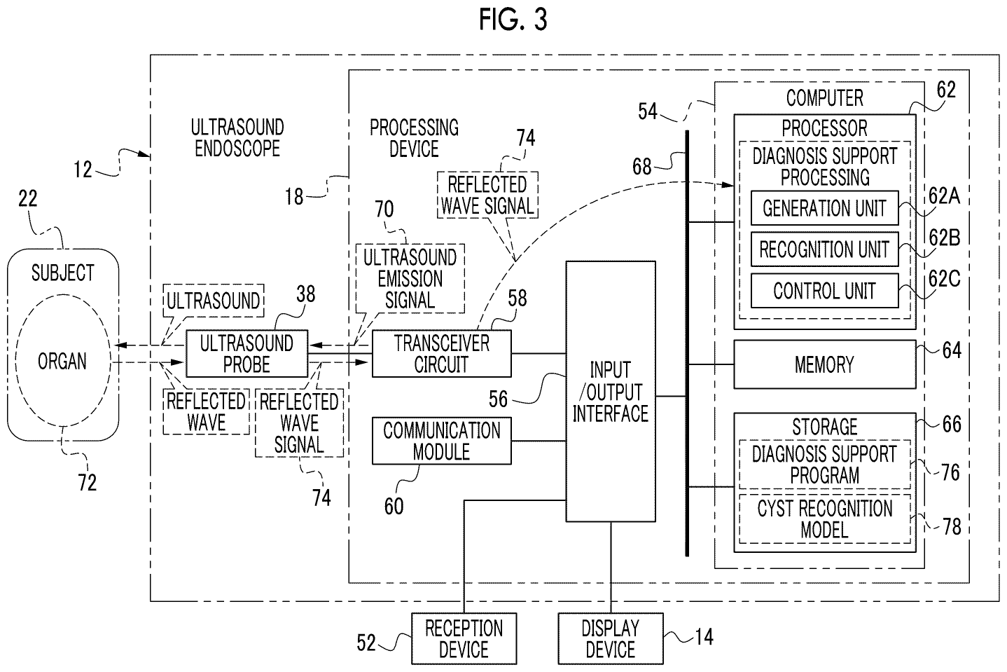

As shown in FIG. 3 as an example, the processing device 18 comprises a computer 54, an input/output interface 56, a transceiver circuit 58, and a communication module 60. The computer 54 is an example of a “computer” according to the present disclosure.

The computer 54 comprises a processor 62, a memory 64, and a storage 66. The input/output interface 56, the processor 62, the memory 64, and the storage 66 are connected to a bus 68.

The processor 62 controls the entire processing device 18. For example, the processor 62 includes a CPU and a GPU, and the GPU is operated under the control of the CPU, and is mainly responsible for executing image processing. It should be noted that the processor 62 may be one or more CPUs with an integrated GPU function or may be one or more CPUs without an integrated GPU function. The processor 62 may include a multi-core CPU or a TPU. In the present embodiment, the processor 62 is an example of a “processor” according to the present disclosure.

The memory 64 is a memory that temporarily stores information, and is used as a work memory by the processor 62. Examples of the memory 64 include a RAM. The storage 66 is a nonvolatile storage device that stores various programs, various parameters, and the like. Examples of the storage 66 include a flash memory (for example, an EEPROM) and/or an SSD. It should be noted that the flash memory and the SSD are merely examples, and the storage 66 may be other nonvolatile storage devices such as an HDD, or may be a combination of two or more types of nonvolatile storage devices.

The reception device 52 is connected to the input/output interface 56, and the processor 62 acquires the instruction received by the reception device 52 via the input/output interface 56 and executes processing in response to the acquired instruction.

The transceiver circuit 58 is connected to the input/output interface 56. The transceiver circuit 58 generates an ultrasound emission signal 70 having a pulse waveform to output the ultrasound emission signal 70 to the ultrasound probe 38 in response to an instruction from the processor 62. The ultrasound probe 38 converts the ultrasound emission signal 70 input from the transceiver circuit 58 into the ultrasound and emits the ultrasound to an organ 72 (for example, a pancreas) as an internal part of the subject 22. The ultrasound probe 38 receives a reflected wave that is obtained by the ultrasound emitted to the organ 72 being reflected at the organ 72, converts the reflected wave into a reflected wave signal 74, which is an electrical signal, and outputs the reflected wave signal 74 to the transceiver circuit 58. The transceiver circuit 58 digitizes the reflected wave signal 74 input from the ultrasound probe 38 and outputs the digitized reflected wave signal 74 to the processor 62 via the input/output interface 56. The processor 62 generates the ultrasound image 24 (see FIG. 1) showing an aspect of the organ 72 based on the reflected wave signal 74 input from the transceiver circuit 58 via the input/output interface 56. In addition, the processor 62 also generates a Doppler image based on the reflected wave signal 74 in parallel with the generation of the ultrasound image 24.

Although not shown in FIG. 3, the illumination device 46 (see FIG. 2) is also connected to the input/output interface 56. The processor 62 controls the illumination device 46 via the input/output interface 56 to change the type of the light applied from the illumination device 46 or to adjust an amount of the light. In addition, although not shown in FIG. 3, the camera 48 (see FIG. 2) is also connected to the input/output interface 56. The processor 62 controls the camera 48 via the input/output interface 56 or acquires the image obtained by imaging the inside of the body of the subject 22 using the camera 48 via the input/output interface 56.

The communication module 60 is connected to the input/output interface 56. The communication module 60 is an interface including a communication processor, an antenna, and the like. The communication module 60 is connected to a network (not shown) such as a LAN or a WAN, and controls communication with an external device (for example, a server, a personal computer, and/or a tablet terminal).

The display device 14 is connected to the input/output interface 56, and the processor 62 controls the display device 14 via the input/output interface 56 such that various types of information are displayed on the display device 14.

The reception device 52 is connected to the input/output interface 56, and the processor 62 acquires the instruction received by the reception device 52 via the input/output interface 56 and executes processing in response to the acquired instruction.

The storage 66 stores a diagnosis support program 76 and a cyst recognition model 78. The processor 62 performs diagnosis support processing by reading out the diagnosis support program 76 from the storage 66 and executing the readout diagnosis support program 76 on the memory 64. The diagnosis support processing is processing of executing recognition processing of recognizing an observation target region from the organ 72 using an AI method, and supporting a diagnosis performed by the user 20 (see FIG. 1) based on a recognition result of the recognition processing. The diagnosis support processing is implemented by the processor 62 operating as a generation unit 62A, a recognition unit 62B, and a control unit 62C in accordance with the diagnosis support program 76 executed on the memory 64.

In the present embodiment, the diagnosis support program 76 is an example of a “program” according to the present disclosure. In addition, the cyst recognition model 78 is a trained model used in the AI method processing of recognizing the cyst 28 shown in the ultrasound image 24. In addition, in the present embodiment, the cyst recognition model 78 is an example of a “trained model” according to the present disclosure.

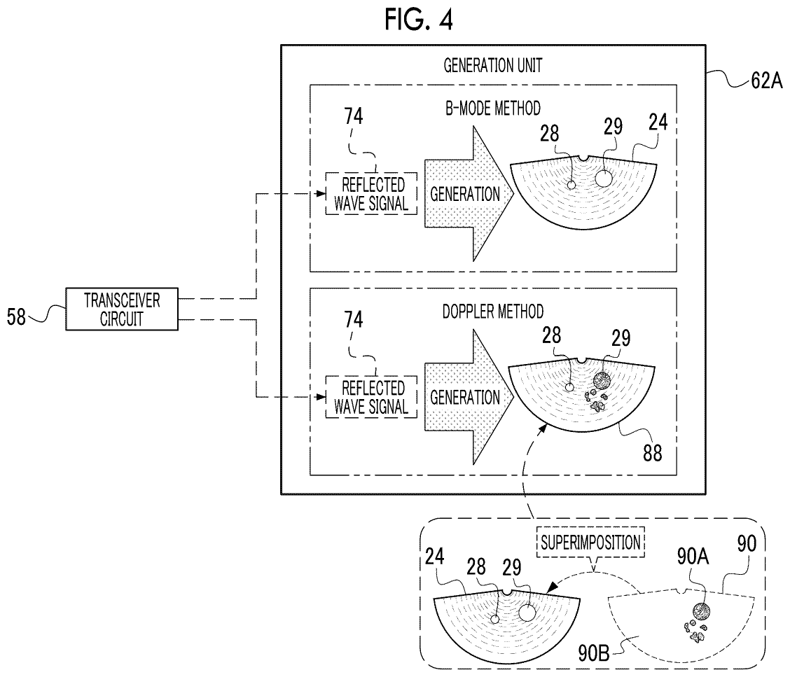

As shown in FIG. 4 as an example, the generation unit 62A acquires the reflected wave signal 74 from the transceiver circuit 58 and generates the ultrasound image 24 using the B-mode method based on the acquired reflected wave signal 74. That is, the generation unit 62A generates a B-mode image based on the reflected wave signal 74 as the ultrasound image 24. The B-mode image generated as the ultrasound image 24 by the generation unit 62A is an image showing a tomogram of the organ 72 (see FIG. 3) in a two-dimensional manner. The example shown in FIG. 4 shows an example in which the ultrasound image 24 in which the cyst 28 and the blood vessel 29 are shown is generated by the generation unit 62A, but as a matter of course, a B-mode image in which the cyst 28 and/or the blood vessel 29 are not shown may be generated as the ultrasound image 24.

In the present embodiment, the ultrasound image 24 generated by the generation unit 62A is an example of an “ultrasound image” according to the present disclosure. In addition, in the present embodiment, the cyst 28 shown in the ultrasound image 24 is an example of an “observation target region of a subject”, a “lesion”, and a “cyst” according to the present disclosure. In addition, in the present embodiment, the blood vessel 29 shown in the ultrasound image 24 is an example of a “blood vessel” according to the present disclosure.

In the present embodiment, in the ultrasound diagnosis on the subject 22, the B-mode method and the Doppler method are used in combination. That is, the generation unit 62A generates a Doppler image 88 in parallel with the ultrasound image 24. Specifically, the generation unit 62A acquires the reflected wave signal 74 used for generating the ultrasound image 24 from the transceiver circuit 58, and generates the Doppler image 88 by the Doppler method based on the acquired reflected wave signal 74. A frame rate of the ultrasound image 24 is higher than a frame rate of the Doppler image 88, and the ultrasound image 24 and the Doppler image 88 are associated with each other in units of a predetermined number of frames of the ultrasound image 24 (for example, in units of 15 frames of the ultrasound image 24). An example of the frame rate of the ultrasound image 24 is 30 frames/second. In addition, an example of the frame rate of the Doppler image 88 is 15 frames/second. It should be noted that this is merely an example, and the frame rate of the ultrasound image 24 may be the same as the frame rate of the Doppler image 88.

The Doppler image 88 is an image obtained by superimposing a color map 90 in which a characteristic (here, as an example, a distribution of blood flow) in the organ 72 is expressed in colors (that is, chromatic colors) on the ultrasound image 24. The color map 90 is information indicating blood flow dynamics specified by using a Doppler effect. In the color map 90, an intensity of the blood flow is expressed in colors. In the present embodiment, a concept of the intensity of the blood flow also includes strength, speed, and/or momentum of the blood flow. The color map 90 includes a blood-flow region 90A indicating a portion where a blood flow is present and a non-blood-flow region 90B indicating a portion where no blood flow is present. From the blood-flow region 90A, the intensity of the blood flow can be specified based on the color applied. Therefore, the image obtained by superimposing the color map 90 on the ultrasound image 24, that is, the Doppler image 88 can also be referred to as a map capable of specifying the intensity of the blood flow. In the present embodiment, the Doppler image 88 is an example of “blood flow distribution information” and a “map” according to the present disclosure.

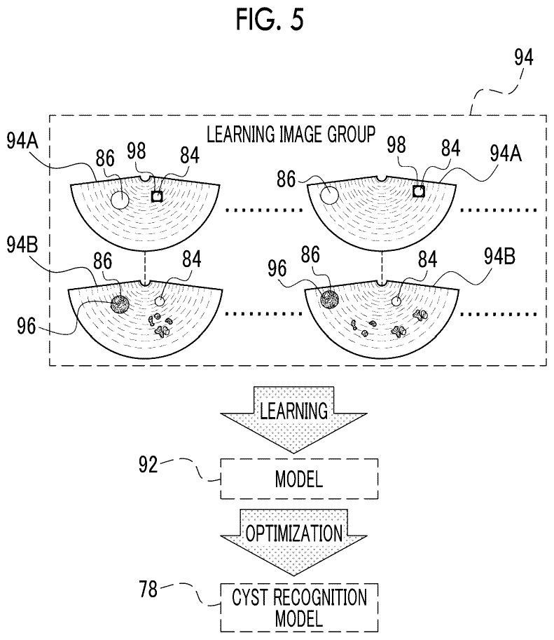

As shown in FIG. 5 as an example, the cyst recognition model 78 is a trained model generated by causing a model 92 to perform machine learning. The cyst recognition model 78 is obtained by performing machine learning for recognizing the cyst 28 (see FIG. 1) shown in the ultrasound image 24 (see FIG. 1). In the machine learning performed on the model 92, a learning image group 94 is used as training data. The learning image group 94 is an image group in which a plurality of different B-mode images 94A and a plurality of different Doppler images 94B having a correspondence relationship with the B-mode images 94A are associated with each other on a one-to-one basis. That is, in the learning image group 94, the Doppler image 94B to be used in combination with the B-mode image 94A is associated with the B-mode image 94A. Examples of the Doppler image 94B used in combination with the B-mode image 94A include an image obtained by superimposing a color map (in other words, a map in which the intensity of the blood flow can be specified in colors) showing the blood flow dynamics of a portion shown in the B-mode image 94A on the B-mode image 94A. Examples of the B-mode image 94A and the Doppler image 94B associated with each other include a sample B-mode image and a sample Doppler image obtained by an actual ultrasound diagnosis, and/or a sample B-mode image and a sample Doppler image generated by so-called generative AI.

Examples of the model 92 include a mathematical model using an NN. Examples of a type of the NN include a YOLO, an R-CNN, and an FCN. In addition, the NN used in the model 92 may be, for example, the YOLO, the R-CNN, or a combination of the FCN and an RNN. The RNN is suitable for machine learning of a plurality of images obtained in time series. It should be noted that the type of the NN described here is merely an example, and other types of the NNs capable of detecting an object by machine learning of an image may also be used.

At least the cyst 84 of the cyst 84 and a blood vessel 86 is shown in the plurality of B-mode images 94A. An annotation 98 is assigned to the B-mode image 94A. The annotation 98 is information capable of specifying a position where the cyst 84 is shown in the B-mode image 94A (for example, information including a plurality of coordinates capable of specifying a position of a rectangular frame circumscribing the cyst 84).

Here, for convenience of description, as an example of the annotation 98, information capable of specifying the position where the cyst 84 is shown in the B-mode image 94A is shown, but this is merely an example. For example, the annotation 98 may include various types of information related to a lesion shown in the B-mode image 94A, such as information capable of specifying a type of the lesion shown in the B-mode image 94A, an importance of the lesion, and/or a size of the lesion. Examples of the lesion include a lesion (for example, a tumor) of a type different from the cyst.

It should be noted that, hereinafter, for convenience of description, processing using the cyst recognition model 78 will be described as processing that is actively performed by the cyst recognition model 78 as a main subject. That is, for convenience of description, the cyst recognition model 78 will be described as having a function of performing processing on input information and outputting a processing result. In addition, hereinafter, for convenience of description, a part of processing of training the model 92 will also be described as processing that is actively performed by the model 92 as a main subject. That is, for convenience of description, the model 92 will be described as having a function of performing processing on input information and outputting a processing result.

The B-mode image 94A and the Doppler image 94B included in the learning image group 82 and associated with each other are input to the model 92 as example data. In response to this, the model 92 recognizes a position where the cyst 84 is shown in the B-mode image 94A based on the input B-mode image 94A and the Doppler image 94B (in other words, predicts the position where the cyst 84 is shown in the B-mode image 94A). Then, the model 92 outputs a recognition result (in other words, a prediction result). The recognition result includes information capable of specifying the position recognized by the model 92 as the position where the cyst 84 is shown in the B-mode image 94A. Examples of the information capable of specifying the position recognized by the model 92 include information including a plurality of coordinates capable of specifying a position of a bounding box surrounding a region predicted as a position at which the cyst 84 is present (that is, the position of the bounding box in the B-mode image 94A).

The model 92 is adjusted according to an error between the annotation 98 assigned as correct answer data to the B-mode image 94A input to the model 92 and the recognition result output from the model 92. That is, the model 92 is optimized by adjusting a plurality of optimization variables (for example, a plurality of connection weights and a plurality of offset values) in the model 92 such that the error is minimized, whereby the cyst recognition model 78 is generated. That is, a data structure of the cyst recognition model 78 is obtained by causing the model 92 to learn the plurality of different B-mode images 94A to which the annotations 98 are assigned and the plurality of Doppler images 94B having a correspondence relationship with the plurality of B-mode images 94A.

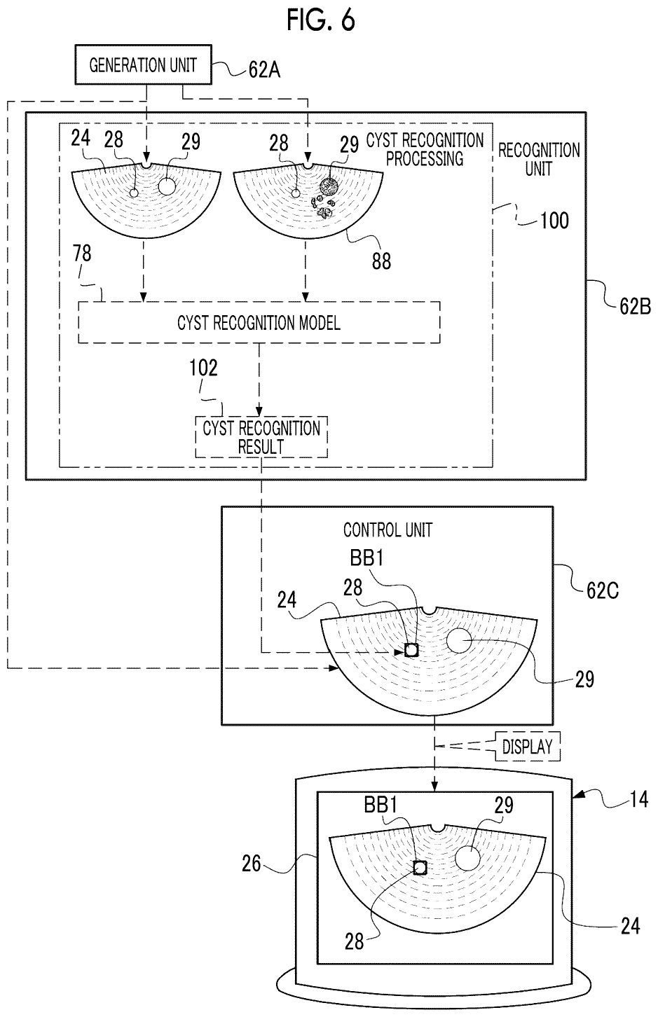

As shown in FIG. 6 as an example, the recognition unit 62B acquires the ultrasound image 24 and the Doppler image 88 which correspond to each other and are generated by the generation unit 62A from the generation unit 62A, and executes cyst recognition processing 100 on the acquired ultrasound image 24 and Doppler image 88. The cyst recognition processing 100 is processing of inputting the ultrasound image 24 and the Doppler image 88 acquired from the generation unit 62A and corresponding to each other to the cyst recognition model 78 to cause the cyst recognition model 78 to recognize the cyst 28. In the present embodiment, the cyst recognition processing 100 is an example of “recognition processing” according to the present disclosure.

A cyst recognition result 102 is generated by the cyst recognition model 78 by executing the cyst recognition processing 100. The cyst recognition result 102 includes information capable of specifying the position of the cyst 28 shown in the ultrasound image 24.

The control unit 62C acquires the cyst recognition result 102 generated by the cyst recognition model 78. The control unit 62C reflects the cyst recognition result 102 on the ultrasound image 24 used in the cyst recognition processing 100. In this case, for example, the cyst recognition result 102 is reflected in the ultrasound image 24 as a bounding box BB1. The bounding box BB1 is superimposed on a portion in the ultrasound image 24 where the cyst 28 is shown, by the control unit 62C. In the example shown in FIG. 6, the bounding box BB1 is superimposed on the ultrasound image 24 as a circumscribing frame for the cyst 28 shown in the ultrasound image 24.

The control unit 62C outputs the ultrasound image 24 in which the bounding box BB1 is superimposed on the portion in the ultrasound image 24 where the cyst 28 is shown. In the example shown in FIG. 6, the ultrasound image 24 on which the bounding box BB1 is superimposed is displayed on the screen 26 by the control unit 62C. That is, the bounding box BB1 superimposed on the ultrasound image 24 is displayed on the screen 26 as information capable of distinguishing the cyst 28 from portions other than the cyst 28.

Here, as an example of outputting the ultrasound image 24 on which the bounding box BB1 is superimposed, an example in which the ultrasound image 24 on which the bounding box BB1 is superimposed is displayed on the screen 26 has been described, but the present disclosure is not limited to this. For example, the ultrasound image 24 on which the bounding box BB1 is superimposed may be stored in a storage region (the storage 66, a server, a personal computer, a tablet terminal, an electronic medical record, or the like), or may be recorded on a medium (for example, paper) by a printer (not shown). In addition, a position of the bounding box BB1 (that is, a position of the cyst 28) in the ultrasound image 24 may be guided by a voice through a speaker (not shown).

In the present embodiment, the bounding box BB1 superimposed on the ultrasound image 24 is an example of a “recognition result” according to the present disclosure. In addition, in the present embodiment, the bounding box BB1 superimposed on the ultrasound image 24 displayed on the screen 26 is an example of “information capable of distinguishing a lesion from a portion other than the lesion” according to the present disclosure.

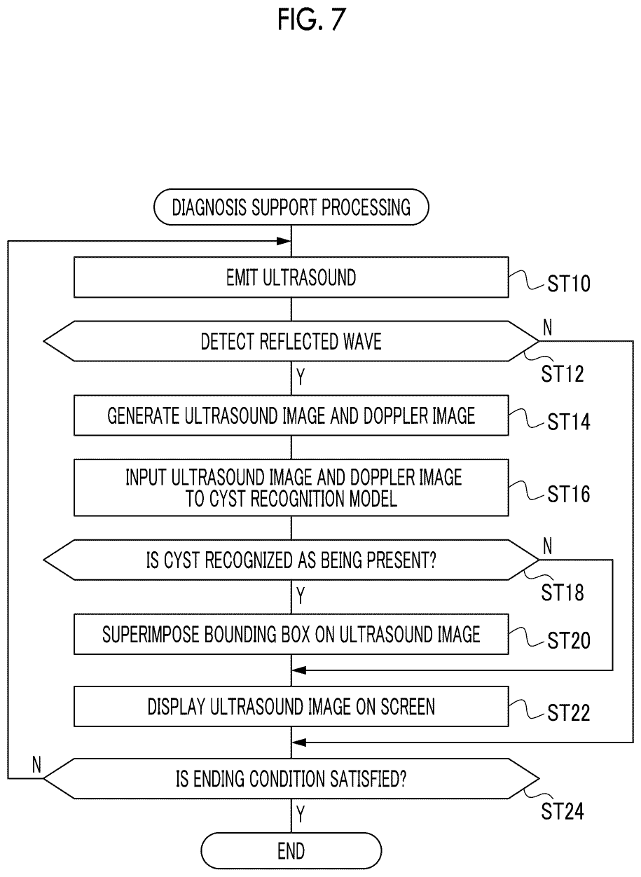

Next, an operation of the endoscope system 10 will be described with reference to FIG. 7.

FIG. 7 shows an example of a flow of the diagnosis support processing performed by the processor 62 of the processing device 18. The flow of the diagnosis support processing shown in FIG. 7 is an example of an “image processing method” according to the present disclosure.

In the diagnosis support processing shown in FIG. 7, first, in step ST10, the generation unit 62A controls the ultrasound probe 38 via the transceiver circuit 58 to cause the ultrasound probe 38 to emit ultrasound to the organ 72 (here, as an example, the pancreas). After the processing of step ST10 is executed, the diagnosis support processing proceeds to step ST12.

In step ST12, the generation unit 62A determines whether or not the reflected wave based on the ultrasound emitted by executing the processing in step ST10 is detected by the ultrasound probe 38. In step ST12, in a case where the reflected wave based on the ultrasound emitted by executing the processing of step ST10 is not detected by the ultrasound probe 38, a negative determination is made, and the diagnosis support processing proceeds to step ST28. In step ST12, in a case where the reflected wave based on the ultrasound emitted by executing the processing of step ST10 is detected by the ultrasound probe 38, a positive determination is made, and the diagnosis support processing proceeds to step ST14.

In step ST14, the generation unit 62A generates the ultrasound image 24 and the Doppler image 88 based on the reflected wave detected by the ultrasound probe 38. After the processing of step ST14 is executed, the diagnosis support processing proceeds to step ST16.

In step ST16, the recognition unit 62B inputs the ultrasound image 24 and the Doppler image 88 generated in step ST14 to the cyst recognition model 78. After the processing of step ST16 is executed, the diagnosis support processing proceeds to step ST18.

In step ST18, the recognition unit 62B determines whether or not the cyst 28 is recognized as being shown in the ultrasound image 24 by the cyst recognition model 78 based on the input of the ultrasound image 24 and the Doppler image 88 to the cyst recognition model 78. In step ST18, in a case where the cyst 28 is not recognized as being shown in the ultrasound image 24, a negative determination is made, and the diagnosis support processing proceeds to step ST24. In step ST18, in a case where the cyst 28 is recognized as being shown in the ultrasound image 24, a positive determination is made, and the diagnosis support processing proceeds to step ST20.

In step ST20, the recognition unit 62B superimposes the cyst recognition result 102 as the bounding box BB1 on the ultrasound image 24 generated in step ST14. After the processing of step ST20 is executed, the diagnosis support processing proceeds to step ST22.

In step ST22, the control unit 62C displays the ultrasound image 24 on which the bounding box BB1 is superimposed on the screen 26. That is, in a case where a negative determination is made in step ST18, the ultrasound image 24 generated in step ST14 is displayed on the screen 26 by executing the processing of step ST26. Furthermore, in a case where the diagnosis support processing has proceeded to step ST22 after the processing of step ST20 is executed, the ultrasound image 24 on which the bounding box BB1 is superimposed is displayed on the screen 26 by executing the processing of step ST22. After the processing of step ST22 is executed, the diagnosis support processing proceeds to step ST24.

In step ST24, the control unit 62C determines whether or not a condition for ending the diagnosis support processing is satisfied. Examples of the condition for ending the diagnosis support processing include a condition in which an instruction to end the diagnosis support processing is received by the reception device 52. In step ST24, in a case where the condition for ending the diagnosis support processing is not satisfied, a negative determination is made, and the diagnosis support processing proceeds to step ST10. In step ST24, in a case where the condition for ending the diagnosis support processing is satisfied, the diagnosis support processing is ended.

As described above, in the endoscope system 10, the ultrasound image 24 obtained by the B-mode method and the Doppler image 88 obtained by the Doppler method used in combination with the B-mode method are input to the cyst recognition model 78 to cause the cyst recognition model 78 to recognize the cyst 28 shown in the ultrasound image 24. The cyst recognition result 102 generated by the cyst recognition model 78 is superimposed on a portion in the ultrasound image 24 where the cyst 28 is shown, as the bounding box BB1. Then, the ultrasound image 24 in which the bounding box BB1 is superimposed on the portion in the ultrasound image 24 where the cyst 28 is shown is displayed on the screen 26.

As a result, compared to a case where the trained model is caused to recognize the cyst 82 shown in the ultrasound image 24 without using the Doppler image 88, the user 20 can visually recognize the image obtained by the B-mode method, that is, a highly accurate recognition result (in the example shown in FIG. 6, the cyst recognition result 102) for the cyst 82 shown in the ultrasound image 24. This means that the user 20 can visually distinguish between the portion in the ultrasound image 24 where the cyst 28 is shown and portions other than the cyst 28 through the bounding box BB1. In addition, this also means that this can contribute to, in a case where the cyst 28 and the blood vessel 29 are shown in the ultrasound image 24 in a state of being mixed, enabling the user 20 to visually distinguish between the cyst 28 and the blood vessel 29 (in other words, it is possible to suppress the blood vessel 29 from being erroneously recognized as the cyst 28 or the cyst 28 from being erroneously recognized as the blood vessel 29).

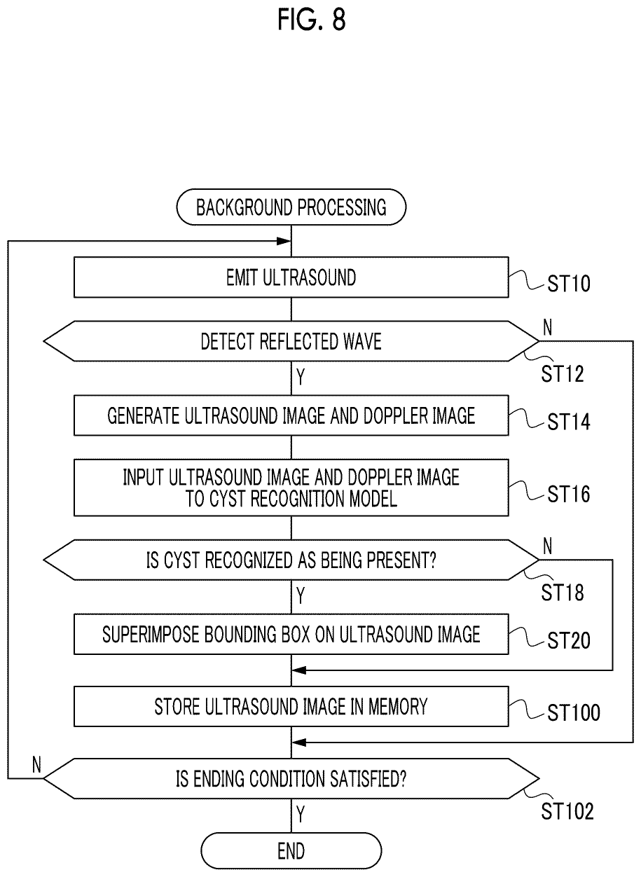

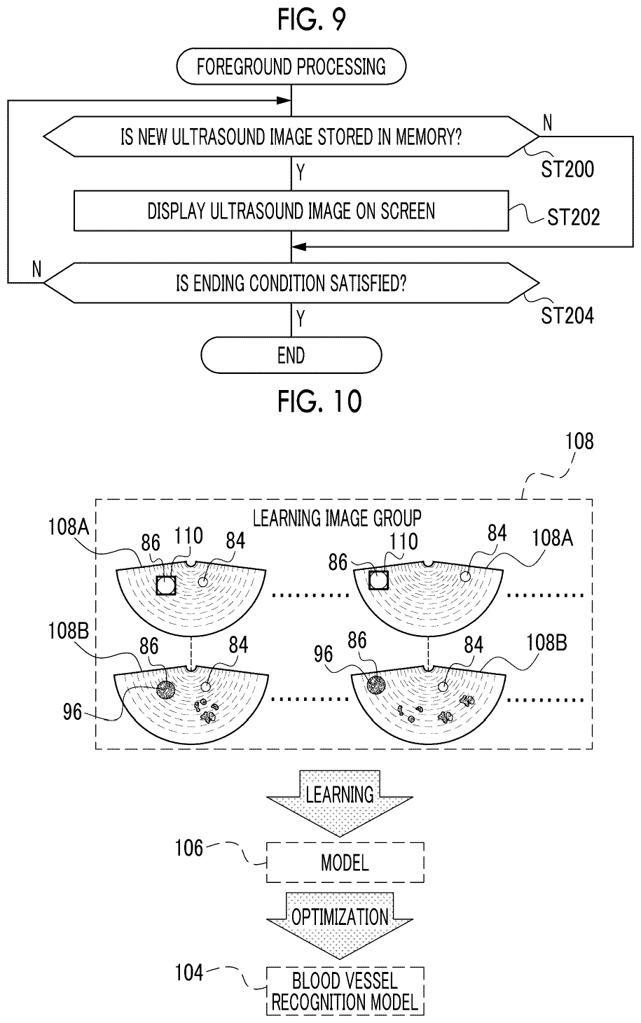

In the above-described embodiment, the form example has been described in which the diagnosis support processing is executed by sequential processing, but the diagnosis support processing may be implemented by parallel processing. For example, as shown in FIGS. 8 and 9, the same processing as the diagnosis support processing described in the above-described embodiment may be implemented by executing background processing and foreground processing in parallel. The background processing refers to processing in a so-called background, and the foreground processing refers to processing in a so-called foreground. For example, in the background processing, the cyst recognition processing 100 is executed, while in the foreground processing, the ultrasound image 24 is displayed on the screen 26.

Here, an example of a flow of the background processing will be described with reference to FIG. 8.

The background processing shown in FIG. 8 is different from the diagnosis support processing shown in FIG. 7 in that processing of step ST100 and processing of step ST102 are included instead of the processing of step ST22 and the processing of step ST24.

In step ST100 shown in FIG. 8, the control unit 62C stores the ultrasound image 24 in the memory 64. The ultrasound image 24 is stored in the memory 64 using a FIFO method. The memory 64 is also a buffer memory that can store a plurality of ultrasound images 24 in time series. In a case where a negative determination is made in step ST18, the processing of step ST100 is executed, and thus the ultrasound image 24 generated in step ST14 is stored in the memory 64. In addition, in a case where the diagnosis support processing proceeds to step ST100 after the processing of step ST22 is executed, the processing of step ST100 is executed, and thus the ultrasound image 24 on which the bounding box BB1 is superimposed is stored in the memory 64. After the processing of step ST100 is executed, the diagnosis support processing proceeds to step ST102.

In step ST102, the control unit 62C determines whether or not a condition for ending the background processing is satisfied. Example of the condition for ending the background processing include a condition in which an instruction to end the background processing is received by the reception device 52. In step ST102, in a case where the condition for ending the background processing is not satisfied, a negative determination is made, and the background processing proceeds to step ST10. In step ST102, in a case where the condition for ending the background processing is satisfied, the background is ended.

Next, an example of a flow of the foreground processing will be described with reference to FIG. 9.

In step ST200 shown in FIG. 9, the control unit 62C determines whether or not a new ultrasound image 24 is stored in the memory 64. In step ST200, in a case where the new ultrasound image 24 is not stored in the memory 64, a negative determination is made, and the foreground processing proceeds to step ST202.

In step ST202, the control unit 62C acquires the ultrasound image 24 stored in the memory 64 (for example, the oldest ultrasound image 24 among the plurality of ultrasound images 24 stored in the memory 64 using the FIFO method) and displays the acquired ultrasound image 24 on the screen 26. After the processing of step ST202 is executed, the foreground processing proceeds to step ST204.

In step ST204, the control unit 62C determines whether or not a condition for ending the foreground processing is satisfied. Examples of the condition for ending the foreground processing include a condition in which an instruction to end the foreground processing is received by the reception device 52. In step ST204, in a case where the condition for ending the foreground processing is not satisfied, a negative determination is made, and the foreground processing proceeds to step ST200. In step ST204, in a case where the condition for ending the foreground processing is satisfied, the foreground is ended.

As described above, in the examples shown in FIGS. 8 and 9, in the background, the cyst recognition processing 100 is executed, while in the foreground, the ultrasound image 24 is displayed on the screen 26. In this way, the user 20 can visually recognize the ultrasound image 24 without perceiving that the cyst recognition processing 100 is being executed.

In the above-described embodiment, the form example has been described in which the cyst 28 is recognized by the cyst recognition model 78, but this is merely an example. For example, as shown in FIGS. 10 to 12, a blood vessel recognition model 104 may be caused to recognize the blood vessel 29.

In order to implement this form example, the blood vessel recognition model 104 shown in FIG. 10 is used instead of the cyst recognition model 78. In the example shown in FIG. 10, the blood vessel recognition model 104 is an example of the “trained model” according to the present disclosure.

The blood vessel recognition model 104 is obtained by performing machine learning for recognizing the blood vessel 29 (see FIG. 1) shown in the ultrasound image 24 (see FIG. 1). As shown in FIG. 10 as an example, the blood vessel recognition model 104 is generated by causing a model 106 to perform machine learning. The model 106 has the same data structure as the model 92.

In the machine learning performed on the model 106, a learning image group 108 is used as training data. The learning image group 108 is an image group in which a plurality of different B-mode images 108A and a plurality of different Doppler images 108B having a correspondence relationship with the B-mode images 108A are associated with each other on a one-to-one basis. That is, in the learning image group 108, the Doppler image 108B to be used in combination with the B-mode image 108A is associated with the B-mode image 108A. Examples of the Doppler image 108B used in combination with the B-mode image 108A include an image obtained by superimposing a color map (in other words, a map in which the intensity of blood flow can be specified in colors) showing the blood flow dynamics of a portion shown in the B-mode image 108A on the B-mode image 108A. Examples of the B-mode image 108A and the Doppler image 108B associated with each other include a sample B-mode image and a sample Doppler image obtained by an actual ultrasound diagnosis, and/or a sample B-mode image and a sample Doppler image generated by so-called generative AI.

At least the blood vessel 86 of the cyst 84 and the blood vessel 86 is shown in the plurality of B-mode images 108A. An annotation 110 is assigned to the B-mode image 108A. The annotation 110 is information capable of specifying a position where the blood vessel 86 is shown in the B-mode image 108A (for example, information including a plurality of coordinates capable of specifying a position of a rectangular frame circumscribing the blood vessel 86).

Here, for convenience of description, as an example of the annotation 110, information capable of specifying the position where the blood vessel 86 is shown in the B-mode image 108A is shown, but this is merely an example. For example, the annotation 110 may include various types of information related to the blood vessel 86 shown in the B-mode image 108A, such as information capable of specifying a type of the blood vessel 86 shown in the B-mode image 108A, an importance of the blood vessel 86, and/or a size of the blood vessel 86.

It should be noted that, hereinafter, for convenience of description, processing using the blood vessel recognition model 104 will be described as processing that is actively performed by the blood vessel recognition model 104 as a main subject. That is, for convenience of description, the blood vessel recognition model 104 will be described as having a function of performing processing on input information and outputting a processing result. In addition, hereinafter, for convenience of description, a part of processing of training the model 106 will also be described as processing that is actively performed by the model 106 as a main subject. That is, for convenience of description, the model 106 will be described as having a function of performing processing on input information and outputting a processing result.

The B-mode image 108A and the Doppler image 108B included in the learning image group 108 are input to the model 106. In response to this, the model 106 recognizes a position where the blood vessel 86 is shown in the B-mode image 108A from the input B-mode image 108A and the Doppler image 108B (in other words, predicts the position where the blood vessel 86 is shown in the B-mode image 108A). Then, the model 106 outputs a recognition result (in other words, a prediction result). The recognition result includes information capable of specifying the position recognized by the model 106 as the position where the blood vessel 86 is shown in the B-mode image 108A. Examples of the information capable of specifying the position recognized by the model 106 include information including a plurality of coordinates capable of specifying a position of a bounding box surrounding a region predicted as a position at which the blood vessel 86 is present (that is, the position of the bounding box in the B-mode image 108A).

The model 106 is adjusted according to an error between the annotation 110 assigned as correct answer data to the B-mode image 108A input to the model 106 and the recognition result output from the model 106. That is, the model 106 is optimized by adjusting a plurality of optimization variables (for example, a plurality of connection weights and a plurality of offset values) in the model 106 such that the error is minimized, whereby the blood vessel recognition model 104 is generated. That is, a data structure of the blood vessel recognition model 104 is obtained by causing the model 106 to learn the plurality of different B-mode images 108A to which the annotations 110 are assigned and the plurality of Doppler images 108B having a correspondence relationship with the plurality of B-mode images 108A.

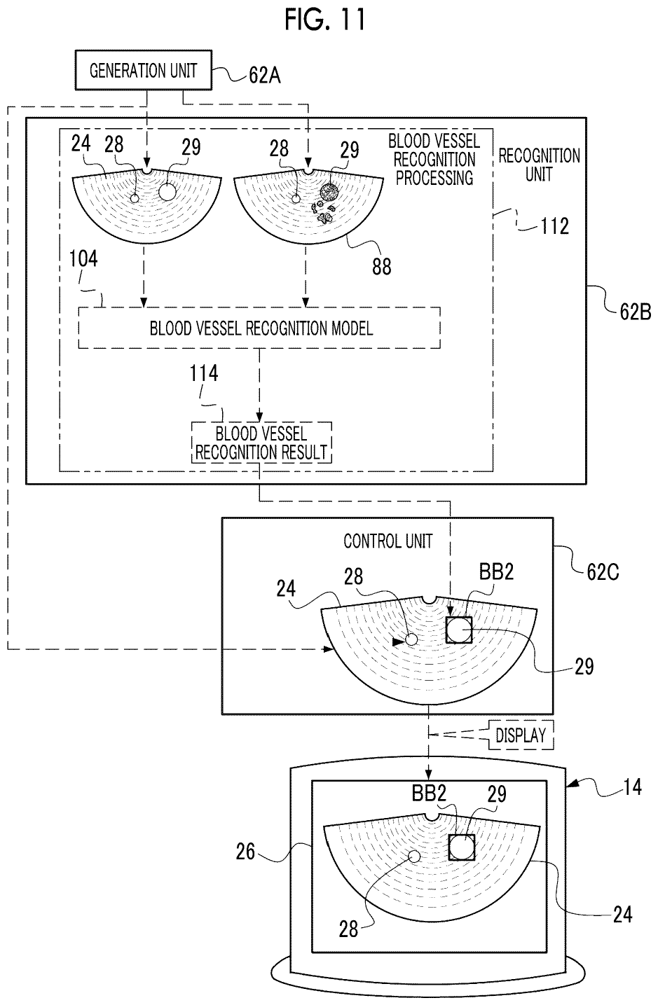

As shown in FIG. 11 as an example, the recognition unit 62B acquires the ultrasound image 24 and the Doppler image 88 which correspond to each other and are generated by the generation unit 62A from the generation unit 62A, and executes blood vessel recognition processing 112 on the acquired ultrasound image 24 and Doppler image 88. The blood vessel recognition processing 112 is processing of inputting the ultrasound image 24 and the Doppler image 88 acquired from the generation unit 62A and corresponding to each other to the blood vessel recognition model 104 to cause the blood vessel recognition model 104 to recognize the blood vessel 29. In the example shown in FIG. 11, the blood vessel recognition processing 112 is an example of the “recognition processing” according to the present disclosure. In addition, in the example shown in FIG. 11, the blood vessel 29 is an example of an “observation target region” and the “blood vessel” according to the present disclosure.

A blood vessel recognition result 114 is generated by the blood vessel recognition model 104 by executing the blood vessel recognition processing 112. The blood vessel recognition result 114 includes information capable of specifying the position of the blood vessel 29 shown in the ultrasound image 24.

The control unit 62C acquires the blood vessel recognition result 114 generated by the blood vessel recognition model 104. The control unit 62C reflects the blood vessel recognition result 114 on the ultrasound image 24 used in the blood vessel recognition processing 112. In this case, for example, the blood vessel recognition result 114 is reflected in the ultrasound image 24 as a bounding box BB2. The bounding box BB2 is superimposed on a portion in the ultrasound image 24 where the blood vessel 29 is shown, by the control unit 62C. In the example shown in FIG. 11, the bounding box BB2 is superimposed on the ultrasound image 24 as a circumscribing frame for the blood vessel 29 shown in the ultrasound image 24.

The control unit 62C outputs the ultrasound image 24 in which the bounding box BB2 is superimposed on the portion in the ultrasound image 24 where the blood vessel 29 is shown. In the example shown in FIG. 11, the ultrasound image 24 on which the bounding box BB2 is superimposed is displayed on the screen 26 by the control unit 62C. That is, the bounding box BB2 superimposed on the ultrasound image 24 is displayed on the screen 26 as information capable of distinguishing the blood vessel 29 from portions other than the blood vessel 29.

Here, as an example of outputting the ultrasound image 24 on which the bounding box BB2 is superimposed, an example in which the ultrasound image 24 on which the bounding box BB2 is superimposed is displayed on the screen 26 has been described, but the present disclosure is not limited to this. For example, the ultrasound image 24 on which the bounding box BB2 is superimposed may be stored in a storage region (the storage 66, a server, a personal computer, a tablet terminal, an electronic medical record, or the like), or may be recorded on a medium (for example, paper) by a printer (not shown). In addition, a position of the bounding box BB2 (that is, the position of the blood vessel 29) in the ultrasound image 24 may be guided by a voice through a speaker (not shown).

In the present embodiment, the bounding box BB2 superimposed on the ultrasound image 24 is an example of the “recognition result” according to the present disclosure. In addition, in the present embodiment, the bounding box BB2 superimposed on the ultrasound image 24 displayed on the screen 26 is an example of the “information capable of distinguishing a blood vessel from a portion other than the blood vessel” according to the present disclosure.

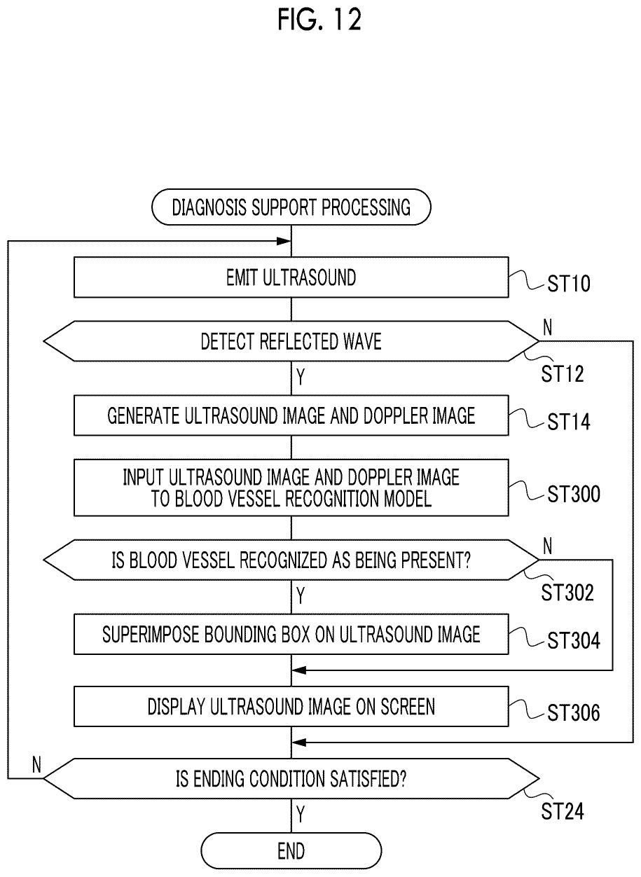

FIG. 12 shows a modification example of the flow of the diagnosis support processing performed by the processor 62 of the processing device 18, that is, an example of a flow of processing of recognizing the blood vessel 29 and suppressing erroneous recognition of the cyst 28 as the blood vessel 29. The flow of the diagnosis support processing shown in FIG. 12 is an example of the “image processing method” according to the present disclosure.

The flowchart shown in FIG. 12 is different from the flowchart shown in FIG. 7 in that processing of steps ST300 to ST306 is provided instead of the processing of steps ST16 to ST22.

In step ST300 shown in FIG. 12, the recognition unit 62B inputs the ultrasound image 24 and the Doppler image 88 generated in step ST14 to the blood vessel recognition model 104. After the processing of step ST300 is executed, the diagnosis support processing proceeds to step ST302.

In step ST302, the recognition unit 62B determines whether or not the blood vessel 29 is recognized as being shown in the ultrasound image 24 by the blood vessel recognition model 104 based on the input of the ultrasound image 24 and the Doppler image 88 to the blood vessel recognition model 104. In step ST302, in a case where the blood vessel 29 is not recognized as being shown in the ultrasound image 24, a negative determination is made, and the diagnosis support processing proceeds to step ST24. In step ST302, in a case where the blood vessel 29 is recognized as being shown in the ultrasound image 24, a positive determination is made, and the diagnosis support processing proceeds to step ST304.

In step ST304, the recognition unit 62B superimposes the blood vessel recognition result 114 as the bounding box BB2 on the ultrasound image 24 generated in step ST14. After the processing of step ST304 is executed, the diagnosis support processing proceeds to step ST306.

In step ST306, the control unit 62C displays the ultrasound image 24 on which the bounding box BB2 is superimposed on the screen 26. That is, in a case where a negative determination is made in step ST302, the ultrasound image 24 generated in step ST14 is displayed on the screen 26 by executing the processing of step ST306. In addition, in a case where the diagnosis support processing has proceeded to step ST306 after the processing of step ST304 is executed, the ultrasound image 24 on which the bounding box BB2 is superimposed is displayed on the screen 26 by executing the processing of step ST306. After the processing of step ST306 is executed, the diagnosis support processing proceeds to step ST24.

As described above, in the examples shown in FIGS. 10 to 12, in the endoscope system 10, the ultrasound image 24 obtained by the B-mode method and the Doppler image 88 obtained by the Doppler method used in combination with the B-mode method are input to the blood vessel recognition model 104 to cause the blood vessel recognition model 104 to recognize the blood vessel 29 shown in the ultrasound image 24. The blood vessel recognition result 114 generated by the blood vessel recognition model 104 is superimposed on the portion in the ultrasound image 24 where the blood vessel 29 is shown, as the bounding box BB2. Then, the ultrasound image 24 in which the bounding box BB2 is superimposed on the portion the ultrasound image 24 where the blood vessel 29 is shown in is displayed on the screen 26.

As a result, compared to a case where the trained model is caused to recognize the blood vessel 29 shown in the ultrasound image 24 without using the Doppler image 88, the user 20 can visually recognize the image obtained by the B-mode method, that is, a highly accurate recognition result (in the example shown in FIG. 11, the blood vessel recognition result 114) for the blood vessel 29 shown in the ultrasound image 24. This means that the user 20 can visually distinguish between the portion in the ultrasound image 24 where the blood vessel 29 is shown and portions other than the blood vessel 29 through the bounding box BB2. In addition, this also means that this can contribute to, in a case where the cyst 28 and the blood vessel 29 are shown in the ultrasound image 24 in a state of being mixed, enabling the user 20 to visually distinguish between the cyst 28 and the blood vessel 29 (in other words, it is possible to suppress the blood vessel 29 from being erroneously recognized as the cyst 28 or the cyst 28 from being erroneously recognized as the blood vessel 29).

Although the form example has been described in which the diagnosis support processing shown in FIG. 12 is executed instead of the diagnosis support processing shown in FIG. 7, this is merely an example. For example, the diagnosis support processing shown in FIG. 7 and the diagnosis support processing shown in FIG. 12 may be performed in parallel. In this case, the bounding box BB1 and the bounding box BB2 may be displayed on the screen 26 in a visually distinguishable manner (for example, color, brightness, thickness, and/or line type). In addition, the bounding box BB1 and the bounding box BB2 may be displayed in a display aspect in which meanings of the bounding box BB1 and the bounding box BB2 are each specifiable. For example, the bounding box BB1 and the bounding box BB2 may be displayed in a display aspect in which the bounding box BB1, which is a bounding box capable of specifying the position of the cyst 28, is distinguished from the bounding box BB2, which is a bounding box capable of specifying the position of the blood vessel 29. In addition, information (for example, an identifier) capable of visually specifying that the bounding box BB1 is information capable of specifying the position of the cyst 28 and the bounding box BB2 is information capable of specify the position of the blood vessel 29 may be displayed.

In the example shown in FIG. 5, the form example has been described in which the machine learning for recognizing the cyst 28 shown in the ultrasound image 24 is performed, and in the example shown in FIG. 11, the form example has been described in which the machine learning for recognizing the blood vessel 29 shown in the ultrasound image 24 is performed, but these are merely examples. For example, machine learning may be performed to recognize the cyst 28 and the blood vessel 29 shown in the ultrasound image 24. In this case, machine learning is performed to recognize the cyst 28 and the blood vessel 29 to be distinguishable from each other. In order to implement this, an annotation capable of specifying the position of the cyst 28 and an annotation capable of specifying the position of the blood vessel 29 may be used as the correct answer data. The ultrasound image 24 and the Doppler image 88 are input to the trained model obtained by performing the machine learning for recognizing the cyst 28 and the blood vessel 29 to be distinguishable from each other, whereby the cyst 28 and the blood vessel 29 are recognized to be distinguishable from each other by the trained model.

In this way, in a case where the cyst 28 and the blood vessel 29 are recognized to be distinguishable from each other by the trained model, the bounding box BB1 can be superimposed on a portion in the ultrasound image 24 where the cyst 28 is shown in the same manner as in the example shown in FIG. 6, and the bounding box BB2 can be superimposed on a portion in the ultrasound image 24 where the blood vessel 29 is shown in the same manner as in the example shown in FIG. 11.

In the above-described embodiment, the trained model for object recognition using a bounding box method with AI has been described as an example, but the present disclosure is also established by using a trained model for object recognition using a segmentation method with AI instead of the trained model for object recognition using the bounding box method with AI or together with the trained model for object recognition using the bounding box method with AI.

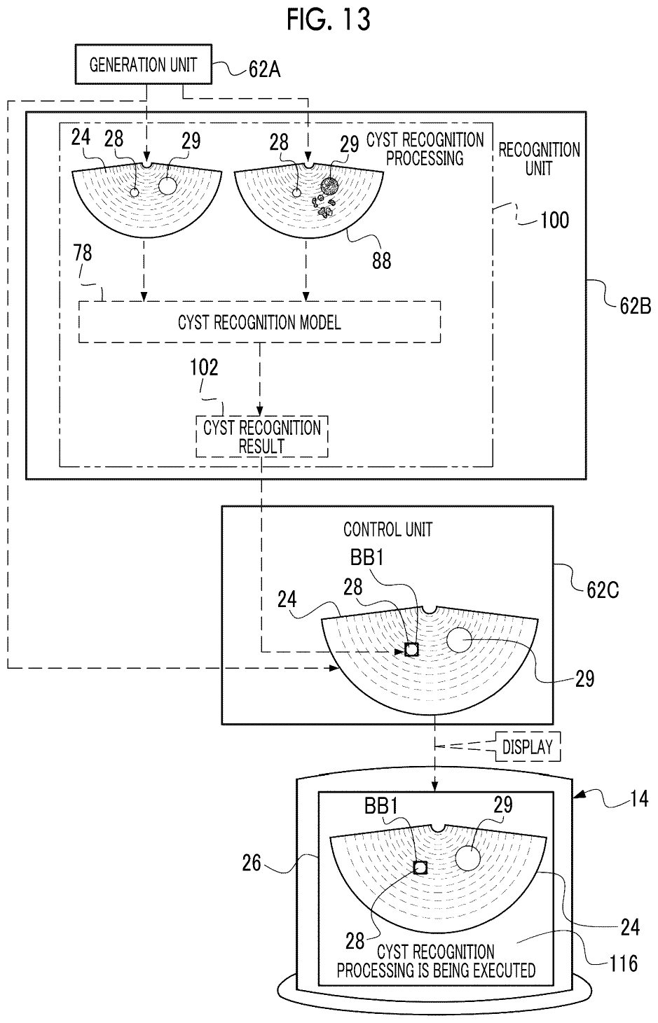

In the above-described embodiment, the form example has been described in which the ultrasound image 24 is displayed on the screen 26. However, for example, as shown in FIG. 13, in a case where the cyst recognition processing 100 is being executed, information 116 indicating that the cyst recognition processing 100 is being executed may be displayed on the screen 26, may be output as a voice, or may be stored in a storage region (the storage 66, a server, a personal computer, a tablet terminal, an electronic medical record, or the like). In the example shown in FIG. 13, as an example of the information 116, text indicating that the cyst recognition processing 100 is being executed is displayed on the screen 26. As a result, the user 20 can recognize that the cyst recognition processing 100 is being executed. In a case where the blood vessel recognition processing 112 is being executed, text indicating that the blood vessel recognition processing 112 is being executed may be displayed on the screen 26.

In addition, in the examples shown in FIGS. 8 and 9, in a case where the background processing is executed, in the foreground processing, information indicating that the background processing (that is, the cyst recognition processing 100 and/or the blood vessel recognition processing 112 in the background) is being executed may be displayed on the screen 26, may be output as a voice, or may be stored in a storage region.

In the above-described embodiment, the generation of the ultrasound image 24 using the B-mode method and the generation of the Doppler image 88 using the Doppler method are performed in parallel, and the ultrasound image 24 and the Doppler image 88 generated at the same time are associated with each other. However, this is merely an example. For example, the generation of the ultrasound image 24 using the B-mode method and the generation of the Doppler image 88 using the Doppler method may be alternately performed, and the ultrasound image 24 and the Doppler image 88 generated in successive frames may be associated with each other.

In the above-described embodiment, the form example has been described in which the ultrasound image 24 and the Doppler image 88 are generated based on the reflected wave detected by the ultrasound probe 38 of the ultrasound endoscope 12, but this is merely an example, and even in a case where the ultrasound image (that is, an image corresponding to the ultrasound image 24) and the Doppler image (that is, an image corresponding to the Doppler image 88) are generated based on the reflected wave detected by the extracorporeal ultrasound probe, the diagnosis support processing can be executed in the same manner as in the above-described embodiment.

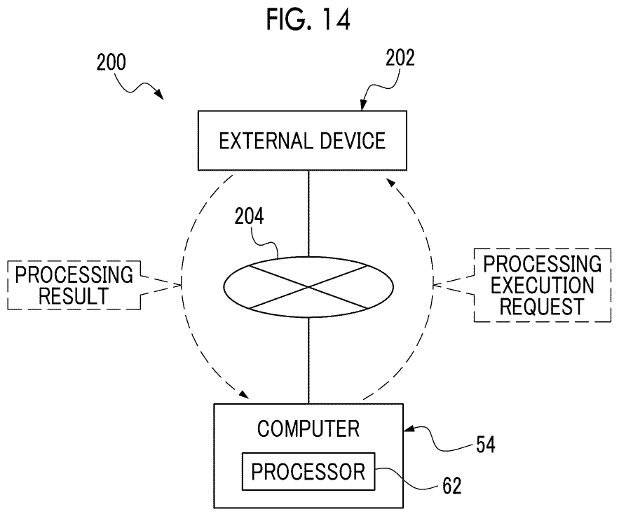

In the embodiment described above, the form example has been described in which the diagnosis support processing is performed by the computer 54, but the present disclosure is not limited to this. At least a part of processing included in the diagnosis support processing may be performed by a device provided outside the computer 54. Hereinafter, an example of this case will be described with reference to FIG. 14.

FIG. 14 is a conceptual diagram showing an example of a configuration of the endoscope system 200. The endoscope system 200 is an example of an “endoscope system” according to the present disclosure. The endoscope system 200 is different from the endoscope system 10 described in the above-described embodiment in that an external device 202 is provided.

The external device 202 is communicatively connected to the computer 54 via a network 204 (for example, a WAN and/or a LAN).

Examples of the external device 202 include at least one server that performs data transmission and reception directly or indirectly with the computer 54 via the network 204. The external device 202 receives a processing execution instruction given from the processor 62 of the computer 54 via the network 204. Then, the external device 202 executes processing according to the received processing execution instruction and transmits a processing result to the computer 54 via the network 204. In the computer 54, the processor 62 receives the processing result transmitted from the external device 202 via the network 204 and executes processing using the received processing result.

Examples of the processing execution instruction include an instruction for the external device 202 to execute at least a part of the diagnosis support processing. A first example of at least a part of the diagnosis support processing (that is, processing executed on the external device 202) includes the cyst recognition processing 100 and/or the blood vessel recognition processing 112 described above. In this case, the external device 202 executes the cyst recognition processing 100 and/or the blood vessel recognition processing 112 in response to the processing execution instruction given from the processor 62 via the network 204, and transmits a recognition result (for example, the cyst recognition result 102 and/or the blood vessel recognition result 114) to the computer 54 via the network 204. In the computer 54, the processor 62 receives the recognition result and executes the same processing as in the above-described embodiment by using the received recognition result.

A second example of at least a part of the diagnosis support processing (that is, processing executed on the external device 202) includes a part of the processing performed by the control unit 62C. An example of a part of the processing performed by the control unit 62C is processing of superimposing the bounding box BB1 or BB2 on the ultrasound image 24. In this case, the external device 202 executes the processing of superimposing the bounding box BB1 or BB2 on the ultrasound image 24 in response to the processing execution instruction given from the processor 62 via the network 204, and transmits a processing result (for example, the ultrasound image 24 on which the bounding box BB1 or BB2 is superimposed) to the computer 54 via the network 204. In the computer 54, the processor 62 receives the processing result and executes the same processing as in the above-described embodiment using the received processing result.

For example, the external device 202 is implemented by cloud computing. It should be noted that the cloud computing is merely an example, and the external device 202 may be implemented by network computing such as fog computing, edge computing, or grid computing. Instead of the server, at least one personal computer or the like may be used as the external device 202. In addition, a computing device having a communication function equipped with a plurality of types of AI functions may be used.

In the above-described embodiment, the form example has been described in which the diagnosis support program 76 is stored in the storage 66, but the present disclosure is not limited to this. For example, the diagnosis support program 76 may be stored in a portable computer-readable non-transitory storage medium, such as an SSD or a USB memory. The diagnosis support program 76 stored in the non-transitory storage medium is installed in the computer 54 of the endoscope system 10. The processor 62 executes the diagnosis support processing according to the diagnosis support program 76.

In addition, the diagnosis support program 76 may be stored in a storage device of another computer, a server, or the like that is connected to the endoscope system 10 via the network. Then, the diagnosis support program 76 may be downloaded and installed in the computer 54 in response to a request from the endoscope system 10.

It is not necessary to store all of the diagnosis support program 76 in a storage device such as another computer or a server device connected to the endoscope system 10 or to store all of the diagnosis support program 76 in the storage 66, and only a part of the diagnosis support program 76 may be stored.

The following various processors can be used as a hardware resource for executing the diagnosis support processing. Examples of the processor include a CPU that is a general-purpose processor that executes software, that is, a program, to function as the hardware resource executing the diagnosis support process. Examples of the processor also include a dedicated electric circuit that is a processor having a dedicated circuit configuration designed to execute specific processing, such as an FPGA, a PLD, or an ASIC. A memory is built in or connected to any processor, and any processor executes the diagnosis support processing using the memory.

The hardware resource for executing the diagnosis support processing may be configured by one of the various processors or by a combination of two or more processors of the same type or different types (for example, a combination of a plurality of FPGAs or a combination of a CPU and an FPGA). The hardware resource for executing the diagnosis support processing may also be one processor.

A first example of the configuration in which the hardware resource is configured by one processor includes a form in which one processor is configured by a combination of one or more CPUs and software, and this processor functions as the hardware resource for executing the diagnosis support processing. As a second example, as typified by a SoC or the like, there is a form in which a processor that implements all functions of a system including a plurality of hardware resources executing the diagnosis support processing with one IC chip is used. As described above, the diagnosis support processing is implemented using one or more of the various processors as the hardware resource.

More specifically, an electric circuit in which circuit elements such as semiconductor elements are combined can be used as a hardware structure of the various processors. In addition, the above-described diagnosis support processing is merely an example. Therefore, it goes without saying that unnecessary steps may be deleted, new steps may be added, or the processing order may be changed within a range that does not deviate from the gist.

The above-described contents and the above-shown contents are the detailed description of the parts according to the present disclosure, and are merely examples of the present disclosure. For example, the description of the configuration, the function, the operation, and the effect are the description of examples of the configuration, the function, the action, and the effect of the parts according to the present disclosure. Accordingly, it goes without saying that unnecessary parts may be deleted, new elements may be added, or replacements may be made with respect to the above-described contents and the above-shown contents within a range that does not deviate from the gist of the present disclosure. In addition, in order to avoid complications and facilitate understanding of the parts according to the present disclosure, the description of common technical knowledge or the like, which does not particularly require the description for enabling the implementation of the present disclosure, is omitted in the above-described contents and the above-shown contents.

All of the documents, the patent applications, and the technical standards described in the present specification are incorporated into the present specification by reference to the same extent as in a case in which the individual documents, patent applications, and technical standards are specifically and individually stated to be described by reference.

The following appendixes are further disclosed with respect to the above embodiment.

### Supplementary Note 1

A training data generation method including: creating example data in which a sample ultrasound image (for example, the B-mode image 94A or 108A), which is a sample of an ultrasound image obtained by a B-mode method in an ultrasound diagnosis for a subject and in which an observation target region of the subject is shown, and sample blood flow distribution information (for example, the Doppler image 94B or 108B), which is a sample of blood flow distribution information obtained by a Doppler method used in combination with the B-mode method in the ultrasound diagnosis, are associated with each other; and generating training data (for example, the learning image group 94 or 108) by associating an annotation (for example, the annotation 98 or 110) that is capable of specifying a portion in the sample ultrasound image where the observation target region is shown with the example data.

### Supplementary Note 2

A trained model generation method including: creating training data (for example, the learning image group 94) in which example data in which a sample ultrasound image (for example, the B-mode image 94A or 108A), which is a sample of an ultrasound image obtained by a B-mode method in an ultrasound diagnosis for a subject and in which an observation target region of the subject is shown, and sample blood flow distribution information (for example, the Doppler image 94B or 108B), which is a sample of blood flow distribution information obtained by a Doppler method used in combination with the B-mode method in the ultrasound diagnosis, are associated with each other is associated with an annotation (for example, the annotation 98 or 110) that is capable of specifying a portion in the sample ultrasound image where the observation target region is shown; and generating a trained model (for example, the cyst recognition model 78 or the blood vessel recognition model 104) by causing a model (for example, the model 92 or 106) to execute machine learning using the training data to optimize the model.

What is claimed is:

1. An image processing device comprising:
a processor,
wherein the processor is configured to:
acquire an ultrasound image which is obtained by a B-mode method in an ultrasound diagnosis for a subject and in which an observation target region of the subject is shown, and blood flow distribution information obtained by a Doppler method used in combination with the B-mode method in the ultrasound diagnosis, and
output a recognition result obtained by executing recognition processing of inputting the ultrasound image and the blood flow distribution information to a trained model to cause the trained model to recognize the observation target region.

2. The image processing device according to claim 1,
wherein the observation target region is a lesion, and
the recognition result is information capable of distinguishing the lesion from a portion other than the lesion.

3. The image processing device according to claim 2,
wherein the lesion is a cyst.

4. The image processing device according to claim 2,
wherein the trained model is obtained by performing machine learning for recognizing the lesion or machine learning for recognizing the lesion and a blood vessel shown in the ultrasound image.

5. The image processing device according to claim 1,
wherein the observation target region is a blood vessel, and
the recognition result is information capable of distinguishing the blood vessel from a portion other than the blood vessel.

6. The image processing device according to claim 5,
wherein the trained model is obtained by performing machine learning for recognizing the blood vessel or machine learning for recognizing a lesion shown in the ultrasound image and the blood vessel shown in the ultrasound image.

7. The image processing device according to claim 1,
wherein the blood flow distribution information is a map capable of specifying an intensity of a blood flow.

8. The image processing device according to claim 1,
wherein the outputting of the recognition result includes displaying the recognition result on a first screen.

9. The image processing device according to claim 1,
wherein the ultrasound image is displayed on a second screen in a foreground, and
the recognition processing is executed in a background.

10. The image processing device according to claim 1,
wherein, in a case where the recognition processing is being executed, the processor is configured to output information indicating that the recognition processing is being executed.

11. An endoscope system comprising:
the image processing device according to claim 1; and
an ultrasound probe that emits ultrasound and detects a reflected wave of the ultrasound in a state in which the ultrasound probe is inserted into a body of the subject,
wherein the ultrasound image and the blood flow distribution information are generated based on the reflected wave detected by the ultrasound probe.

12. An image processing method comprising:
acquiring an ultrasound image which is obtained by a B-mode method in an ultrasound diagnosis for a subject and in which an observation target region of the subject is shown, and blood flow distribution information obtained by a Doppler method used in combination with the B-mode method in the ultrasound diagnosis; and
outputting a recognition result obtained by executing recognition processing of inputting the ultrasound image and the blood flow distribution information to a trained model to cause the trained model to recognize the observation target region.

13. A non-transitory computer-readable storage medium storing a program executable by a computer to execute a process comprising:
acquiring an ultrasound image which is obtained by a B-mode method in an ultrasound diagnosis for a subject and in which an observation target region of the subject is shown, and blood flow distribution information obtained by a Doppler method used in combination with the B-mode method in the ultrasound diagnosis; and
outputting a recognition result obtained by executing recognition processing of inputting the ultrasound image and the blood flow distribution information to a trained model to cause the trained model to recognize the observation target region.
